(19) United States
(12) Patent Application Publication
(10) Pub. No.: US 2015/0064672 A1
(43) Pub. Date: Mar. 5, 2015

(54) SYSTEMS AND METHODS FOR TREATMENT OF SUBSTANCE ADDICTION

(71) Applicant: Matthew Bars, Mahwah, NJ (US)

(72) Inventor: Matthew Bars, Mahwah, NJ (US)

(21) Appl. No.: 14/477,929

(22) Filed: Sep. 5, 2014

Related U.S. Application Data
(60) Provisional application No. 61/873,973, filed on Sep. 5, 2013.

(51) Int. Cl. G09B 5/00; G01N 33/493; G01N 33/49; H04L 12/58; G01N 33/497

(52) U.S. Cl. CPC G09B 5/00; H04L 51/36; G01N 33/4972; G01N 33/4925; G01N 33/493
USPC 434/236

(57) ABSTRACT

Systems and methods for treatment for addiction to a substance according to a predetermined action plan that includes a message control center for sending information to a messaging-accessible device associated with a user, an analyzer for analyzing an indicia of use of said substance, and a personalized treatment engine to receive an output from the analyzer and to direct the message control center based, at least in part, on the output from the analyzer and the predetermined action plan for the user.

### CROSS-REFERENCE TO RELATED APPLICATION

This application claims priority under 35 U.S.C. 119(e) from U.S. provisional patent application 61/873,973 having a filing date of Sep. 5, 2013, which is entitled “Systems and Methods for Treatment of Substance Addiction”, which is hereby incorporated by reference.

### FIELD OF THE INVENTION

The present invention relates to systems and methods for substance addiction cessation and therapy, including systems and methods for cigarette smoking cessation and cigarette smoking cessation therapy.

### BACKGROUND

Smoking is an example of a substance addiction (tobacco addiction) that has a high cost to society. Despite the known health dangers, smoking persists among a large portion of addicted individuals. Recent studies demonstrate that while the vast majority of smokers want to quit, historically less than one in ten are successful on their own, and less than a third (31.7%) have used evidence-based methods. See, e.g., Rigotti N A. Treatment of tobacco use and dependence. N Engl J Med 2002; 346(7):506-512. Yet, despite the challenges, in 2008 global sales of stop smoking aids were $3.5 billion and are expected to increase significantly over time.

Historically, the most common measure of smoking is subjective self-assessment, either through global reports or timeline follow-back (TLFB). Each of these depends critically on participants' abilities to accurately remember and recall their own behavior for the course of a day or several days, and in the case of TLFB, with the help of detailed prompts. Despite their wide use, these methods have been shown to be inaccurate due to a number of different cognitive biases.

For example, cigarette smokers tend to display a “digit bias,” or “heap” their reports of smoking around particular numbers reflecting the number of cigarettes per pack or even fractions thereof (e.g., 10 or 20) even though research finds that no such heaping occurs in actual smoking behavior (Klesges, Debon, & Ray, 1995). In one recent study, actual cigarette counts differed by +/−5 to 9 cigarettes per day (CPD) for 43% of smokers and by +/−10 or more CPD for 29% of smokers (Blank, Breland, & Cobb, 2013).

There is widespread agreement that self-reported number of cigarettes and/or cigars smoked is an imprecise measure of tobacco smoke consumption, and offers little to engage smokers in quitting. Accordingly, a need exists for treatment programs that incorporate accurate smoking assessment by patients who desire to reduce or quit smoking or tobacco use altogether.

Recall of the situational cues for smoking has also been shown to be inaccurate (Shiffman, 1993). Further, some of these biases may be particularly strong in smokers who are attempting to quit because the emotional antecedents and consequences of relapse can potentiate recall bias (Shiffman et al., 1997). These limitations of recall have led to the consensus that other methods are necessary for accurately assessing smoking and other health-relevant behaviors across time (Collins & Graham, 2002; Pierce, 2009).

Clinicians and smokers both prefer to see real numbers that assess their addiction, level of tobacco consumption, and progress towards quitting. Providing immediate assessment of an individual's tobacco consumption with biochemical confirmation of reduction in consumption prior to cessation and finally total abstinence can validate a smoker's progress.

Expired breath carbon monoxide determinations have been used to assess severity of nicotine addiction, tobacco cessation and progress toward quitting. For example, in 1999, the National Health Service in England established a national network of Stop Smoking Services. They recommend carbon monoxide assessment of all smokers who start tobacco treatment. In the most recent year for which data is available, April 2010 to March 2011, 9.8% of England's 8 million smokers were treated. Expired breath carbon monoxide was assessed on 95% of those receiving treatment services totaling over 748,000 smokers annually.

Concerns about tobacco withdrawal symptoms (not feeling “normal” when quitting), concerns about living life without cigarettes, and fear of failure prompt most smokers to continue smoking. However, the idea of “cutting down” or reduction in tobacco consumption helps smokers overcome this “cessation anxiety.” Recent research supports the safety and efficacy of FDA-approved tobacco treatment medications in helping smokers reduce their cigarette consumption. Without question, it is safe and effective to use these medications while still smoking. Further, medical science has conclusively demonstrated that multiple tobacco treatment medications used concurrently can increase quit rates in recalcitrant smokers.

Recent research, meta-analyses, and clinical experience demonstrate that combination tobacco treatment pharmacotherapies and Reduction-to-Cessation (Reduce then Quit) protocols can reduce cessation anxiety, minimize withdrawal symptoms, significantly increase quit attempts, and abstinence. An unmet need exists for assisting the tobacco dependent user to participate actively in their treatment anytime, anywhere.

### BRIEF SUMMARY OF THE INVENTION

One aspect of the present invention provides a system for treatment for addiction to a substance according to a predetermined action plan that includes a message control center for sending information to a messaging-accessible device associated with a user; an analyzer for analyzing an indicia of use of said substance, and a personalized treatment engine to receive an output from the analyzer and to direct the message control center based, at least in part, on the output from the analyzer and the predetermined action plan for that user.

Another aspect of the present application provides a method for treatment for addiction to a substance according to a predetermined action plan that includes analyzing a sample from a user for an indicia of substance use via an analyzer; sending output from the analyzer to a personalized treatment engine; and sending information to an messaging-accessible device associated with a user, said information based, at least in part, on output from the analyzer and the predetermined action plan for that user.

Certain embodiments of the present invention are based, at least in part on the combination of a CO analyzer used in conjunction with an interactive action plan for smoking cessation that includes messaging functionality to aid, assist and support the user who desires to quit, reduce, or at least assess their smoking.

One aspect of the present invention provides a system for aiding smoking cessation or smoking reduction therapy according to a predetermined action plan that includes a message control center for sending information to a messaging-accessible device associated with a user, a CO analyzer for analyzing carbon monoxide content of the user's expired breath, and a personalized treatment engine to receive an output from the CO analyzer and to direct the message control center based, at least in part, on the output from the CO analyzer and the predetermined action plan for that user.

Another aspect of the present invention provides a method for aiding smoking cessation or smoking reduction therapy according to a predetermined action plan that includes analyzing carbon monoxide content of the user's expired breath via a CO analyzer, sending output from the CO analyzer to a personalized treatment engine, and sending information to an messaging-accessible device associated with a user, said information based, at least in part, on output from the CO analyzer and the predetermined action plan for that user.

Still another aspect of the present invention provides a system to communicate CO levels from a user's expired breath to a third party that includes a CO analyzer for analyzing carbon monoxide content of the user's expired breath; and a communications gateway to communicate outputs from the CO analyzer to the third party. A method to communicate CO levels from a user's expired breath to a third party including analyzing the carbon monoxide content of the user's expired breath; and communicating the results of the CO analyzer to the third party is also provided. Other methods are understood from the description of the systems of the present invention and their use.

In addition to comprehensive systems and methods to assist smoking cessation programs, the instant invention also provides novel treatment regimens. Another aspect of the present invention provides a method for increasing the efficacy of a nicotine replacement product that includes administering an antacid to a user, and shortly after administering the antacid, administering the nicotine replacement product to the user.

### BRIEF DESCRIPTION OF THE DRAWINGS

FIG. 1 depicts an exemplary and non-limiting system for aiding substance abuse treatment according to one particular embodiment of the present invention

FIG. 2 depicts an exemplary and non-limiting system for aiding smoking cessation or smoking reduction therapy according to one particular embodiment of the present invention.

FIG. 3 depicts an exemplary and non-limiting system to communicate CO levels from a user's expired breath to a third party according to one particular embodiment of the present invention.

FIG. 4 depicts a screenshot from a “Tobacco Warriors-Smoke the Zombies” game.

### DETAILED DESCRIPTION OF THE INVENTION

As used herein, the term “messaging-accessible device” refers to any device that can receive electronic messages from a third party. Electronic messages can include, for example, emails and text messages and can be transmitted, for example, via one or more of land-line, cellular, wireless and satellite-enabled infrastructure. Messaging-accessible devices include traditional desktops and laptops (e.g., PCs and Macs), and also includes smartphones (e.g., iPhones®, Blackberries®, Android® based devices), traditional cellular or mobile phones capable of sending and receiving text messages (sometimes referred to as “dumb phones” or “feature phones”) and tablet computers (e.g., iPads®, Galaxy® tablets).

As used herein, the term “mobile messaging-accessible device” refers to a subset of messaging-accessible devices that are lightweight (e.g., less than three pounds, preferably less than two pounds), and designed such that they are suitable, and intended by the manufacture, to be easily transported by the average user of the device as they leave their homes. Mobile messaging-accessible devices include smartphones, traditional cellular or mobile phones and tablet computers, but do not include traditional desktop and laptops. In general, traditional keyboards (i.e., non-capacitive, non-touch screen keyboards which instead contain raised keys that are of a size such that the well-trained typist would typically rest at least eight fingers on separate “home” keys on the keyboard while typing a message) are not found on mobile messaging-accessible devices, or at least not normally found without special adaptation. Nevertheless, email and text messaging functionality is found on mobile messaging-accessible devices.

As used herein, the term “smartphone” refers to a subset of mobile messaging-accessible devices that include the ability to access the internet along with phone and text messaging functionality and include IPhones®, Android®-based devices (e.g., Samsung Galaxy® devices with voice phone calling functionality) and Blackberry® devices. An example of such a smartphone is disclosed in U.S. Pat. No. 7,356,361.

As used herein a “nicotine replacement product” refers to any product that, when administered, replaces, or seeks to replace, nicotine that would otherwise be obtained from smoking or other orally consumed tobacco products (e.g., smokeless tobacco products such as chewing tobacco and snuff). Examples of nicotine replacement products include, but are not limited to, nicotine gum (e.g., Nicorette®), nicotine transdermal patches (e.g., Nicoderm®), nicotine lozenges (e.g., Commit®). An example of such a patch is disclosed in U.S. Pat. No. 6,224,897. While associated the health risks are not completely known at present, electronic cigarettes can also be considered a nicotine replacement product and can find use within the presently disclosed methods and systems, depending on, for example, the judgment of the health professional that directs the pre-determined action plan. An example of such an electronic cigarette is disclosed in U.S. Pat. No. 8,156,944.

As used herein, “smoking avoidance medication treatment” refers to any protocol involving administration of a prescribed or over the counter product containing an active agent that ameliorates the desire or physical need (perceived or actual) to smoke or otherwise intake nicotine from tobacco products, including both oral (e.g. nicotine gum or lozenges) and transdermally (e.g., nicotine patches) dosage forms. Smoking avoidance medication treatment includes, but is not limited to, administration of nicotine replacement products, as defined above. Smoking avoidance medication treatment further includes prescription drugs, administered as indicated or off label, that reduce the urge to intake nicotine, such as, but not limited to, varenicline (Chantix®), bupropion (Zyban®), nortriptyline (Sensoval®), clonidine (Kapvay®), and cytosine. Smoking avoidance medication treatment is an example of a pre-determined action plan, described in greater detail below.

As used herein, a “CO analyzer” is generally used to refer to any mechanism that can ascertain and output carbon monoxide levels, determined either directly or indirectly, or at least indicate if the carbon monoxide levels ascertained are sufficiently high to indicate that the user has smoked. CO analyzers include, but are not limited to, carbon monoxide sensors that indicate for the presence of carbon monoxide based on the electrochemical changes observed (e.g. voltage produced) when carbon monoxide oxidizes to carbon dioxide. Other forms of sensors can also be employed with the presently described methods and systems, such as sensors that ascertain and output carbon monoxide levels based on infrared-based mechanisms or lab on a chip technology. Output from CO analyzers, or any other analyzer used in the presently disclosed methods and systems, can be communicated wirelessly or via standard “wired” formats (e.g. via computers networded via standard landline or broadband communication protocols).

As used herein, the term “EMR platform” refers to a method of communicating results obtained from the CO analyzer to a party authorized by the user who is trying to quit or reduce their smoking, such a professional (e.g., internal medicine doctor or other physician, psychiatrist, psychologist, tobacco treatment specialist, addictionologist, Physician's Assistant, Nurse Practitioner, etc.) trained in smoking cessation techniques who is working with the user, or representative or custodian of a wellness plan, medical care center, hospital, insurance company/carrier, affordable care organization (ACO)₌website or application. In one embodiment, the results can be conveyed to the authorized party in the form of an electronic medical record in a manner than conforms with patient confidentiality requirements (e.g., HIPAA-compliant).

As used herein, the term “communications gateway” refers to any instrumentality, or assembly of instrumentalities, which can transmit output from a CO analyzer to a third party.

As used herein, the term “antacid” refers to any orally administerable product, including liquids and solid dosage forms that, when administered, serve to reduce the acidity of the buccal mucosa.

As used herein, the term “pre-determined action plan” refers to any previously established or novel protocol that is employed (or administered), in whole or in part, to a user wishing to stop smoking, reduce their smoking, or at least become aware of their current smoking levels, or have others become aware of their current smoking levels. A predetermined action plan for a user can include one or more of, for example, smoking avoidance medication treatment, social support (e.g., via social media platforms or support groups), diversion techniques (e.g., via gaming platforms), user rewards programs (e.g. via a rewards platform). The pre-determined action plan can also include novel treatment regimens, including the novel treatment regimens disclosed herein.

A pre-determined action plan generally includes any protocol or treatment philosophy implemented by a clinician skilled in the art of substance abuse treatment (e.g., a clinician trained in smoking cessation treatment). In certain embodiments, the pre-determined action plan is individualized based on the specific needs and circumstances of the user who wishes to quit smoking. In alternative embodiments, the individualized pre-determined action plan can optionally be supplemented, in whole or in part, by prior protocols that have been evaluated by the present system itself based on the success of prior users (e.g., via machine learning). Certain protocols or modifications of protocols can be presented to the personalized treatment engine based on, for example, recorded “successes” achieve by similar users in the past.

The pre-determined action plan can be any protocol to stop or reduce smoking, and is not limited to the particular, exemplary action plans set forth herein. As implemented by the personalized treatment engine, and other components of the presently described systems, the pre-determined action plan can take advantage of, for example, GPS-enabled devices (e.g. smartphones), NFC (near field communications) buying capability, and social media platforms. For example, in an embodiment that employs a rewards program as part of the pre-determined action plan, GPS-enabled smart devices can detect if a user is near a convenience store or entering a restaurant contiguous to mealtime and transmit a message to beware of and fight smoking urges, and offer rewards points for avoiding or conquering these situations. Buying virtual goods or real FDA-approved medications, particularly if purchased using NFC technology, could also increase reward points. Blowing into the CO analyzer and having a low or lower value than previous values could also award the user points, as could answering questions, or posting on a social media site. Players could also solicit points (donations) from Facebook or other social network friends to purchase virtual or real products including FDA tobacco treatments medications or other items.

As used herein, the term “addiction” refers to both physical and psychological dependence and is used broadly to refer to any chronic use of a substance that, upon sober reflection of that user, is deemed by that user to be unhealthy, excessive, unproductive and/or harmful to the user or the user's acquaintances, or the user's responsibilities.

The present application is directed to systems and methods to treat all substance addictions or to otherwise treat patients who desire, yet cannot stop, using a substance, including, but not limited to, treatment for alcohol, nicotine, marijuana, hashish, cocaine, heroin, amphetamine, methamphetamine, heroin, opium, opioids, MDMA, LSD, anabolic steroid and prescription drug abuse. Solely for purpose of convenience, and not limitation, the present application is described in the specific context of smoking cessation and/or reduction treatment, yet the principles of the present application can equally be applied to other forms of substance abuse.

For example, in the context of smoked drugs, such as cocaine (crack), methamphetamine, opium, marijuana, and particularly cigarettes and cigars, use of a CO analyzer is described. The present application encompasses, however, use of other analyzers depending on the substance at issue. In the context of alcohol use, BAC (blood alcohol concentration) analyzers can be used, such as automatic blood alcohol concentration analyzers commercially available, for example, from YL Instrument Co. (Kyounggi-do, South Korea).

The “lab on a chip” space is particularly suitable for analyzers, as used in the present application. For example, systems like the UltraFAIMS available from Owlstone (Cambridge, UK) can be employed to analyze drug metabolites that can serve as evidence (or non-evidence) of substance use and thus ascertain the user's compliance with the goals of the pre-determined action plan. In the context of smokeless tobacco use, or even in the context of smoking treatment, nicotine or nicotine metabolite analyzers can be used as a replacement for, or in conjunction with, CO analyzers. Other analyzers, which can assay for substance use from expired breath—often by assaying for metabolites of the substance of interest—are disclosed, for example in U.S. Published Application No. 2007/0224128, U.S. Pat. Nos. 7,547,285, and 8,491,494, all of which hereby incorporated by reference in their entirety.

In addition to pre-determined action plans, systems and methods of the present invention also employ carbon monoxide monitors. Carbon monoxide monitors can be used as motivational tools by smoking cessation professionals. Monitoring a person's breath CO levels indicates their level of nicotine dependency, recent consumption, smoking reduction, or abstinence. Results can be accessed by both the individual and the clinician as part of the ongoing smoking cessation process. Embodiments of the presently disclosed systems and methods offer a new, revolutionary method to engage many more smokers and help them conquer their addiction to tobacco.

The analyzers for use in accordance with the methods and systems of the presently disclosed application (e.g., CO analyzer) can be in various forms or shapes. In one particular embodiment, the analyzer (e.g., CO analyzer) is in a form such that it is easily transported by the user as the user engages in their daily activities. In other words, in certain embodiments, the analyzer (e.g., CO analyzer) is easily carried by the user throughout the user's non-sleeping hours. For example, in one embodiment the analyzer (e.g., CO analyzer) is preferably in a size capable of being carried in the user's pocket (e.g., sized such that it comfortable fits in a standard sized pant, shirt or jacket pocket). In one embodiment, the analyzer (e.g., CO analyzer) is adapted to be carried on a key chain, or alternatively, adapted to be worn on the user's belt or waist (e.g., via a clip), or still alternatively, adapted to be worn around the user's neck as a neckless. Alternatively, the analyzer (e.g., CO analyzer) can be adapted to be affixed to the user's clothing or anywhere on the user's body.

In certain non-limiting embodiments, the CO analyzer is further provided with a pressure sensor which can analyze the smoker's exhaled breath duration and intensity of the exhaled breath to ensure the the quality of the obtained sample.

The analyzers for use in accordance with the methods and systems of the presently disclosed application (e.g., CO analyzer) can, in certain, non-limiting embodiments, contain an authentication feature. In one embodiment, the user's identity can be confirmed by providing the analyzer (e.g., CO analyzer) and/or the personalized treatment engine with the ability to recognize the user's facial features via use of biometric facial recognition data and software. Such biometric facial recognition data and software can also incorporate GPS, and data and time stamping. Alternatively, the user's identity can be indentified via one or more of fingerprint data, DNA data or other unique feature that can be ascertained and confirmed by the analyzer and/or personalized treatment engine.

The U.S. Public Health Service's Clinical Practice Tobacco Treatment Guideline has identified common elements of successful counseling techniques for smoking cessation. These include problem solving and skill training, including identifying events, internal states, and activities associated with smoking. Methods and systems of the present invention permits identification of various triggering events in real-time and may be geocoded with GPS coordinates, facilitating the detailing of specific locations where the smoker is prone to smoke. Consistent with evidence based guidelines, a mobile application of the present invention helps the smoker anticipate problem times and places, avoid temptation and smoking trigger situations, and reducing exposure to smoking cues, in real time-anytime, anywhere as it occurs.

An alternative to retrospective measures is a form of real-time, real-world reporting of behavior is referred to as ecological momentary assessment (EMA). Key features of the EMA class of methods include (1) data collection in an ecologically valid environment, (2) real-time or brief interval assessment, and (3) repeated measurements across time and contexts (Shiffman, Stone, & Hufford, 2008). For example, cigarettes per day can be calculated without recall using EMA by instructing smokers to push a button or voice activate and record smoking on a digitable cigarette log on a portable pocket-sized device (e.g., smartphone) each time they smoke. Cigarette counts derived using this method track actual and biological measures of smoking more reliably than recall methods and do not display digit bias (Shiffman, 2009).

EMA offers a number of additional advantages for studying smokers, smoking, and smoking cessation. Smokers may not always be aware of the extent to which factors such as stress, mood, or urges to smoke impact their smoking (Todd, 2004). In effect, subtle or invisible influences on smoking are less likely to be noticed or deemed relevant. EMA overcomes this problem by allowing researchers to measure potential moderators in real time and calculate their relation to smoking.

Real-time assessment of tobacco consumption, as provided by the present invention, allows assessment of the severity of tobacco addiction and biochemical confirmation of reduction in consumption prior to cessation as well as total abstinence. In certain embodiments, the user employs a smart phone. Smart phones can be an ideal delivery mechanism for the tailored interventions of the present invention because of the low cost and the ubiquitous hardware most people already possess.

The messaging functionality is also an integral feature to various embodiments of the presently disclosed systems and methods. Messages can be delivered nearly instantaneously into real-world situations. The presently disclosed invention can include texting functionality to offer evidence-based tobacco treatment techniques, including, but not limited to, problem time identification, coping strategies, and medication therapy adherence reminders.

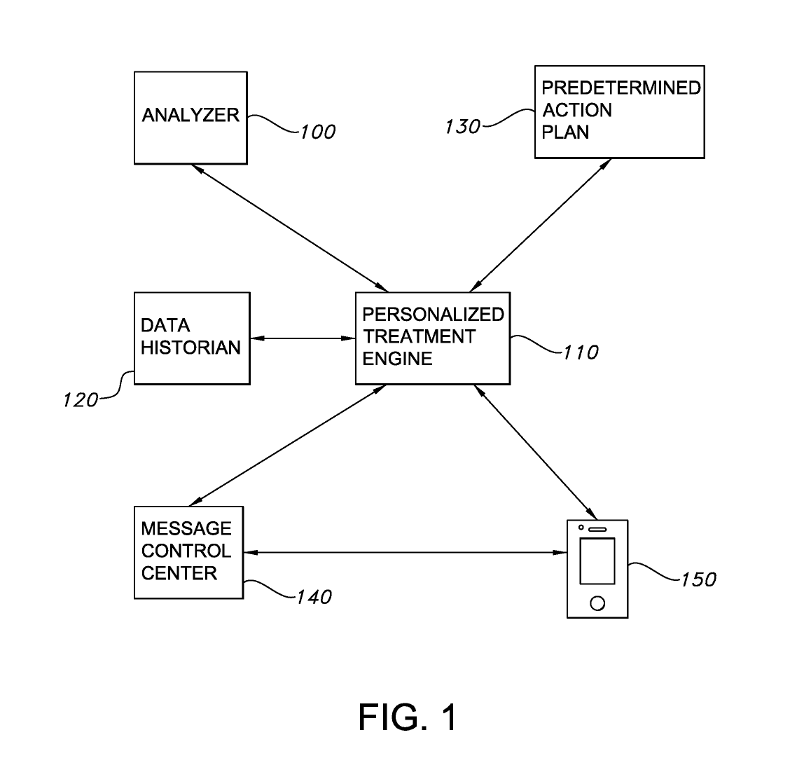

A system for treatment for addiction to a substance according to one non-limiting embodiment is set forth in FIG. 1. An analyzer (100) which can analyze an indicia of use of the relevant substance (e.g, an analyzer that can assay for metabolites of a drug) is provided. Results of the assay can be manually recorded and sent to a personalized treatment engine (110). Alternatively, the analyzer can be provided with wireless capability, in which the results of the assay are automatically sent to the personalized treatment engine. The results can be stored in a data historian (120) which is in communication with the personalized treatment engine.

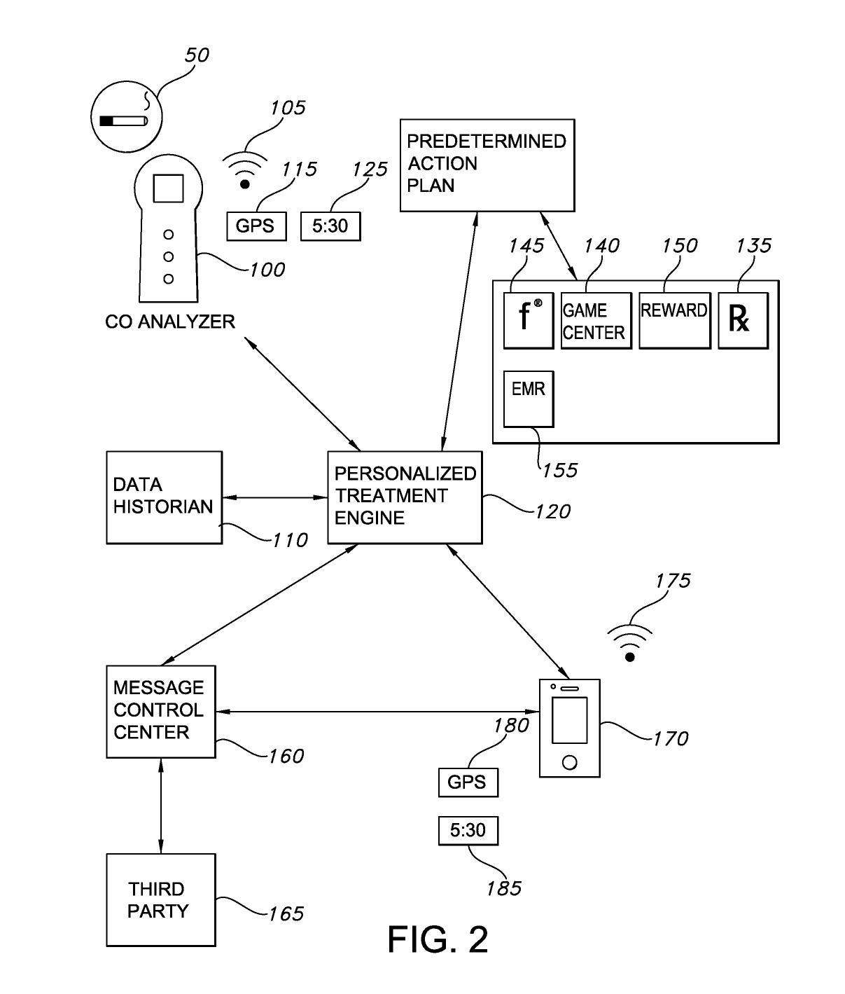

Based on the particular details of the predetermined action plan (130) and the output from analyzer, the personalized treatment center can direct the message control center (140) to interface with the user, which in this non-limiting embodiment is by way of the user's smartphone (150). The interaction can be via a dedicated “app” on the user's smartphone, or alternatively the message control center can direct SMS text messages or emails to the user. Further details regarding the predetermined action plan, personalized treatment engine and message control center are described below in FIG. 2 in the context of smoking cessation, but are equally applicable here for use in the treatment of various forms of substance abuse.

A system for aiding smoking cessation or smoking reduction therapy according to one non-limiting embodiment is set forth in FIG. 2 for purposes of illustration and not limitation. A CO analyzer (100) is provided to measure the CO content in a user's expired breath to determine if the user has been smoking (50). In this embodiment, the CO analyzer is capable of wirelessly communicating (105) output that is stored in a data historian (110), associated with a personalized treatment engine (120), which is discussed below. Output from CO analyzer, in this embodiment, is location stamped (115) via GPS technology based on the location of the reading, and also time stamped (125), and this information, along with the CO content in the users' breath is stored in the data historian.

The CO analyzers for analyzing the carbon monoxide content of a user's expired breath have been employed in the past and are known in the art. See, e.g., Nicotine and Tobacco Research, 4(2), 149-159 (2002), hereby incorporated by reference. For example, CO analyzers are available from KWJ Engineering, Eco-sure, Parallax, Honeywell, and other vendors that can communicate wirelessly (e.g., via Bluetooth® LE compatible), or via traditional wire-based platforms.

In addition to receiving direction based on output from the CO analyzer, the personalized treatment engine (120) is also directed by a predetermined action plan (130). The predetermined action plan can include any established or trial technique(s) employed to prevent smoking and includes prescription drug administration (135), diversion techniques such as smoking-cessation related gaming platforms (140), support services, such as that can be obtained from social media platforms (145) including, but not limited to, Facebook®, Twitter®, LinkedIn®, Nicotine Anonymous® and other message forums where those that are attempting to quit smoking can share their successes, frustrations and challenges and otherwise receive support from like-situationed individuals.

The predetermined action plan, and hence the personalized treatment engine, can also include, for example, a rewards platform (150) to reward users. For example, a user can receive virtual tokens, credits or other virtual or real consideration in exchange for posting negative or relatively lower CO results. An EMR platform (155) can also be provided as part of the predetermined action plan, in which results obtained from the CO Analyzer are communicated, via the message control center (160), to an authorized third party (165), such as a smoking cessation professional working with the user to quit or reduce smoking.

The specifics of the predetermined action plan for a particular user wishing to quit or reduce smoking, non-limiting exemplary features described above, or input into and used to configure the personalized treatment engine (120). The personalized treatment engine serves as the “muscle” to the instantly described system for aiding smoking cessation or smoking reduction therapy in which the desired details of the user's predetermined action plan (e.g. use of rewards platforms, gaming platforms, medications,₌etc.) configured such that they can be communicated to the user's mobile messaging-accessible device (170) (e.g., a smartphone), either directly, or via the message control center (160). In this particular embodiment, the mobile messaging-accessible device is provided with wireless communication (175), GPS (180), and time stamping (185) functionality.

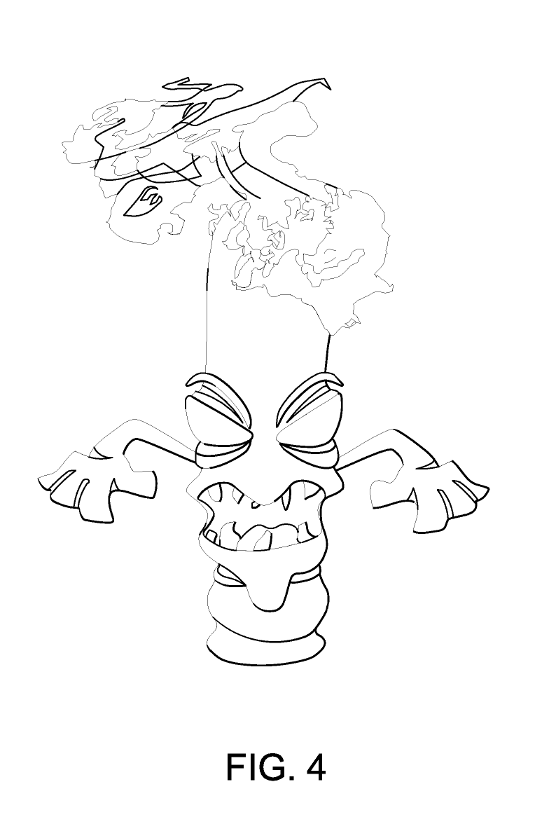

For example, a users' predetermined action plan can include a gaming platform (140) in which it is desired to have the user participate in a “Tobacco Warriors-Smoke the Zombies” game (alternatively titled: “Tar Wars”). In this game, ominous looking cigarettes and evil, zombie-like tobacco executives “attack” the player, the player defending against these threats in a “point and shoot” game, in which the player defend themselves with various weapons, including handguns, automatic weapons (M4, MP5, etc.) and shotguns. In one embodiment, the player can acquire additional lives, weapons or other enhancements by submitting an additional CO sample, by registering a lower CO result (i.e., reflecting reduced smoking) from the CO analyzer, or by inputting additional tobacco use personal health information. A screen shot of the “Tobacco Warriors” game is depicted in FIG. 4.

The game itself can be, for example, stored in the personalized treatment engine or elsewhere in the cloud or remote servers and directly downloaded to the user's mobile messaging-accessible device (170). Alternatively, the personalized treatment can instruct the message control center to send the user information about where the game (which can be housed outside the personalized treatment engine) can be downloaded and played by the user.

It is noted that the “Tobacco Warriors-Smoke the Zombies” game can also be used a recruitment tool to have new users enlist with the presently described methods and systems. For example, the game could be provided as a free “app” on Apple and Droid smartphone and tablet platforms, with advertisements to join the presently described system. Alternatively, the game could initially be provided free, but require the user to enlist in the presently described system in order to ensure continued access to the game.

The personalized treatment engine (120) also serves as an intermediary between the CO analyzer and the user. For example, the personalized treatment engine can be configured, based on the predetermined action plan, to instruct the message control center to send a text message to the user after having received specified output from the CO analyzer. In one non-limiting embodiment, the personalized treatment engine can instruct the message control center to automatically generate a text message providing a congratulatory message to the user who “posts” a pre-determined string of negative or reduced CO analyzer results (i.e., no or reduced CO in user's expired breath) and/or a condolence message to the user who posts a positive (i.e., CO in user's breath) or increased CO result. Thus, in this non-limiting example, the personalized treatment engine evaluates the results received from the CO analyzer and directs the message control center based on those results and the protocol established according to the predetermined action plan.

The message control center (160) can automatically generate text messages, for example, based on software instructions received from the personalized treatment engine (120). For example, the personalized treatment engine can be configured such that output from CO analyzer is sent to the user, via the message control center, and stored along with prior readings, for example, in the user's smartphone or in the cloud or remote servers for later review.

Both the message control center and personalized treatment engine can be processors located within the same computer system or server, or may communicate with each other from separate computer systems or servers. The software instructions can, for example, generate the content of the text message information (e.g., based on a content generation engine) and the intended target of the text message information). In certain embodiments, the system computer system can insert personal information such as the user's first name into the text message to provide some personalization to the message. The personalized treatment engine can be employed to automatically generate such text messages, via the message control center, and may be periodically updated to reflect current trends or information.

Hardware and software to operate the personalized treatment engine and the message control center can be obtained as a stand alone or modified and implemented by commercial vendors who offer, for example SMS servers often used for sending text messages and other information to groups of people. SMS servers can be provided, for example, by IceWarp (Springfield, Va.), Trumpia (Anaheim, Calif.), Ozeki (Debrecen, Hungary), and a multitude of other service providers who can tailor the message control center to work with the personalized treatment engine in accordance with the present disclosure. In certain non-limiting embodiments, messages can be delivered via email, texts and/or phone calls at the user's discretion. Phones can be automated by an IVR system or other or delivered by a human health coach.

The presently disclosed platform delivers a treatment plan via the personalized treatment engine consistent with state of the art medical science, provides medication reminders via the above mentioned message control center, and tracks clinical effectiveness such as reductions in tobacco consumption, changes (if any) in carbon monoxide or other tobacco metabolites and attenuation of tobacco withdrawal symptoms. The software and hardware platform permit sharing the consumption, reduction and/or quitting process with others via preexisting social networks (Facebook, Google Plus, etc) and allows data visualization to the smoker's clinical network (physicians or other health care providers, ACOs, care givers, and significant others). The personalized treatment engine can modify the treatment plan based on smoker success or lack of success. The personalized treatment engine can also be directed in whole or in part by another clinician. The presently disclosed system also enables the real-time or near real time recording of when and where smoking occurs and queries the smoker regarding their smoking behaviors and contiquious activities (e.g.: with coffee, workbreaks, aftermeals, etc.).

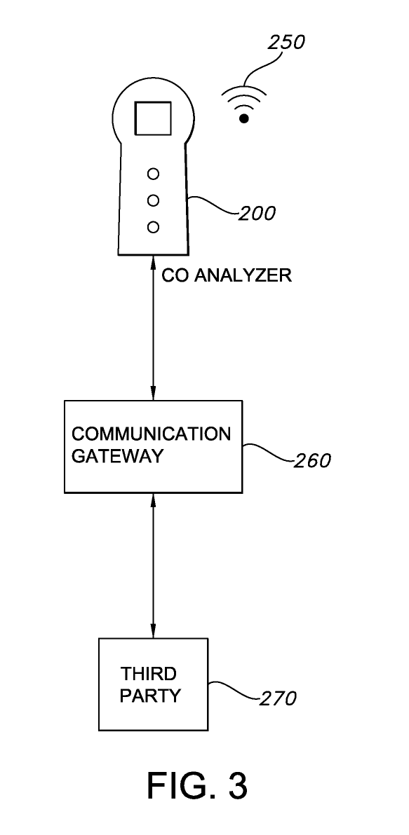

A system to communicate CO levels from a user's expired breath to a third party according to one non-limiting embodiment is set forth in FIG. 3 for purposes of illustration and not limitation. A CO analyzer (200) is provided, in this particular embodiment, with wireless communication (250) capability to communicate output from the analysis to a communications gateway (260). The communication gateway receives the wireless output from the CO analyzer and transmits the data to a third party (270) in real-time, or near real-time. The third party may be, for example, a research entity, a call center or other entities that already offer smoking cessation treatment. It is noted that, with reference to FIG. 2, the combination of the personalized treatment engine and message control center can collectively serve as the communications gateway, and can be considered as such, for communicating results from the CO analyzer to a third party.

For purposes of illustration and not limitation, the CO output can be designed to communicate with a HIPAA compliant server in real time, or near real time. In instances where an internet connection between the CO analyzer and the HIPAA compliant server is lacking, the analyzer can store the output and transmit the same after an internet connection has been established.

The presently disclosed invention also includes novel protocols to stop smoking, which can be used alone, are as part of the above-described systems for aiding smoking cessation or smoking reduction therapy (i.e., as part of the pre-determined action plan). One embodiment of the present application provides a method for increasing the efficacy of a nicotine replacement product that includes administering an antacid to a user, and administering the nicotine replacement product to the user. The nicotine replacement product is preferably administered shortly after administering the antacid. For example, the nicotine replacement product can be administered, within 1 hour of the antacid, within 30 minutes of the antacid, within 15 minutes of the antacid, within 5 minutes of the antacid, or most preferably, within 30 seconds of administering the antacid, or less.

The antacid can be, for example, an OTC medication. In one embodiment, the antacid contains an active agent selected from from one or more of sodium bicarbonate, calcium carbonate, potassium bicarbonate, bismuth subsalicylate, magnesium hydroxide, aluminum hydroxide, and magnesium hydroxide. As it is desired to reduce the acidity of the buccal mucosa, dosage forms that have significant residence time in the mouth region, such as liquid or chewable dosage forms, are preferred, although any dosage form that serves to reduce the acidity of the buccal mucosa when administered can be employed.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should also be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

All publications and references provided herein, which are each hereby incorporated by reference in their entirety, are provided solely for their disclosure prior to the filing date of the present application.

1. A system for treatment for addiction to a substance according to a predetermined action plan comprising:
(a) a message control center for sending information to a messaging-accessible device associated with a user;
(b) an analyzer for analyzing an indicia of use of said substance, and
(c) a personalized treatment engine to receive an output from the analyzer and to direct the message control center based, at least in part, on the output from the analyzer and the predetermined action plan for the user.

2. The system of claim 1, wherein the substance is selected from alcohol, nicotine, marijuana, hashish, cocaine, heroin, amphetamine, methamphetamine, heroin, opium, opioids, MDMA, LSD, anabolic steroids and prescription drugs.

3. The system of claim 2, wherein the substance is nicotine.

4. The system of claim 3, wherein the analyzer is capable of determining amounts of one or more of carbon monoxide, nicotine, cotinine, nicotine N′-oxide, nornicotine, nicotine isomethonium ion, 2-hydroxynicotine and nicotine glucuronide in a users expired breath, blood, urine, saliva, sputum or aromatic vapors from a user.

5. (canceled)

6. (canceled)

7. A method for treatment for addiction to a substance according to a predetermined action plan comprising:
(a) analyzing a sample from a user for an indicia of substance use via an analyzer;
(b) sending output from the analyzer to a personalized treatment engine; and
(c) sending information to an messaging-accessible device associated with a user, said information based, at least in part, on output from the analyzer and the predetermined action plan for user.

8. The method of claim 7, wherein the sample is obtained from the user's expired breath, blood, urine, saliva, sputum, hair or aromatic vapors emanating from the user.

9. A system for aiding smoking cessation or smoking reduction therapy according to a predetermined action plan comprising:
(a) a message control center for sending information to a messaging-accessible device associated with a user;
(b) a CO analyzer for analyzing carbon monoxide content of the user's expired breath, and
(c) a personalized treatment engine to receive an output from the CO analyzer and to direct the message control center based, at least in part, on the output from the CO analyzer and the predetermined action plan for the user.

10. The system of claim 9, wherein the messaging-accessible device is a mobile messaging-accessible device.

11. The system of claim 10, wherein the personalized treatment engine further comprises a data historian to store past outputs from the CO analyzer.

12-15. (canceled)

15. The system of claim 10, wherein the predetermined action plan for the user includes a gaming platform to make available to the user one or more games associated with smoking cessation or smoking reduction, information regarding the gaming platform optionally being introduced to the user via the message control center.

16. The system of claim 10, wherein the predetermined action plan for the user includes a social media platform to connect the user with one or more social media outlets, information regarding the social media platform being introduced to the user via the message control center.

17. The system of claim 10, wherein the predetermined action plan for the user includes a rewards platform to reward the user based, at least in part, on the output received from the CO analyzer, information regarding the reward platform being introduced to the user via the message control center.

18. The system of claim 10, wherein the predetermined action plan for the user includes smoking avoidance medication treatment, information regarding the smoking avoidance medication treatment being sent to the user via the message control center.

19. The system of claim 11, wherein the predetermined action plan for the user includes an EMR platform for securely sharing information obtained by the data historian with one or more third parties previously approved by the user, said securely shared information being transmitted via the message control center.

20. The system of claim 9, wherein the CO analyzer contains a sleep mode, and is capable of activating upon being held by the user, or blown into by the user.

21. The system of claim 9, wherein the system is capable of uniquely identifying the user.

22. The system of claim 21, wherein said identification is achieved via one or more of facial recognition data, fingerprints, DNA or other biometrics uniquely associated with the user.

23. The system of claim 21, wherein said identification is achieved by providing the CO analyzer with a specific and randomly generated pattern of LED on the CO analyzer which is recorded by video or still camera for later verification.

24. The system of claim 9, wherein the personalized treatment engine receives input from a voice recorder log, wherein smoking behavior is recorded by the user's voice.

25. The system of claim 9, wherein the CO analyzer is capable of being carried on a keychain, being clipped to a belt or waistband, worn around the user's neck, worn as an adhesive on clothing or anywhere on the user's body, or being carried in a user's pocket.

26-42. (canceled)
